(19) United States
(12) Patent Application Publication
(10) Pub. No.: US 2020/0129220 A1
(43) Pub. Date: Apr. 30, 2020

(54) CRYOBALLOON FOR INTRAVASCULAR CATHETER SYSTEM

(71) Applicant: Boston Scientific Scimed Inc, Maple Grove, CA (US)

(72) Inventor: Eugene J. Jung, San Diego, CA (US)

(21) Appl. No.: 16/692,363

(22) Filed: Nov. 22, 2019

Related U.S. Application Data
(63) Continuation of application No. PCT/US2018/032580, filed on May 14, 2018.
(60) Provisional application No. 62/510,047, filed on May 23, 2017.
(60) Provisional application No. 62/651,146, filed on Mar. 31, 2018.

(51) Int. Cl. A61B 18/02; A61L 29/08; A61L 29/14

(52) U.S. Cl. CPC A61B 18/02; A61L 29/085; A61B 2018/00255; A61B 2018/0212; A61L 29/14

(57) ABSTRACT

A balloon catheter for an intravascular catheter system includes an outer inflatable balloon and an inner inflatable balloon that is positioned substantially within the outer inflatable balloon. At a nominal working balloon pressure, the outer inflatable balloon has an outer balloon diameter and the inner inflatable balloon has an inner balloon diameter that is greater than the outer balloon diameter at the nominal working balloon pressure. The inner balloon diameter can be at least approximately 5%, 10%, 15%, 20%, 25% or 30% greater than the outer balloon diameter. The inner balloon diameter can be between approximately 29-35 millimeters, and the outer balloon diameter can be between approximately 23-29 millimeters. The inner inflatable balloon can be less compliant than the outer inflatable balloon. The outer balloon compliance can be at least approximately 2%, 5%, 8%, 10%, 15% or 20% greater than the inner balloon compliance.

### CROSS-REFERENCE TO RELATED APPLICATION

This application is a continuation of International Application No. PCT/US18/032580 filed on May 14, 2018 and entitled “CRYOBALLOON FOR INTRAVASCULAR CATHETER SYSTEM”, which claims the benefit of U.S. Provisional Application No. 62/510,047, filed on May 23, 2017, entitled “LOW PROFILE DOUBLE BALLOON CATHETER”, and U.S. Provisional Application No. 62/651,146, filed on Mar. 31, 2018, entitled “VARIABLE-DIAMETER COMPLIANT BALLOON FOR CRYOGENIC BALLOON CATHETER SYSTEM”. As far as permitted, the contents of International Application No. PCT/US18/0325, U.S. Provisional Application No. 62/510,047 and U.S. Provisional Application No. 62/651,146 are incorporated in their entirety herein by reference.

### TECHNICAL FIELD

The present disclosure relates to medical devices and methods for electrophysiology procedures. More specifically, the invention relates to a cryoablation balloon for use in cryoablation procedures to treat cardiac arrythmias.

### BACKGROUND

Cardiac arrhythmias involve an abnormality in the electrical conduction of the heart and are a leading cause of stroke, heart disease, and sudden cardiac death. Treatment options for patients with arrhythmias include medications, implantable devices, and catheter ablation of cardiac tissue.

Catheter ablation involves delivering ablative energy to tissue inside the heart to block aberrant electrical activity from depolarizing heart muscle cells out of synchrony with the normal conduction pattern of the heart. The energy delivery component of the system is typically at or near the most distal (farthest from the operator) portion of the catheter, and often at the tip of the device. Various forms of energy are used to ablate diseased heart tissue. These can include radio frequency (RF), balloon cryotherapy which uses cryoballoons, ultrasound, electroporation (pulsed DC electric fields), and/or laser energy, to name a few. The tip of the catheter is positioned adjacent to targeted tissue, at which time energy is delivered to create tissue necrosis, rendering the ablated tissue incapable of conducting electrical signals. The dose of energy delivered is a critical factor in increasing the likelihood that the treated tissue is permanently incapable of electrical conduction. At the same time, delicate collateral tissue, such as the esophagus, the bronchus, and the phrenic nerve surrounding the ablation zone can be damaged and can lead to undesired complications. Thus, the operator must finely balance delivering therapeutic levels of energy to achieve intended tissue necrosis, while avoiding excessive energy leading to collateral tissue injury.

Atrial fibrillation (AF), one of the most common arrhythmias, can be treated using balloon cryotherapy. In the earliest stages of the disease, paroxysmal AF, the treatment strategy involves isolating the pulmonary vein(s) from the left atrial chamber of the heart. Recently, the use of balloon cryotherapy procedures to treat AF has increased. In part, this stems from ease of use, shorter procedure times, and improved patient outcomes. Ablation of the muscle tissue, located in the atrial chamber of the heart, which is adjacent to the ostium (or opening) of the pulmonary vein can be accomplished using cryoballoon ablation therapy. When a cryoballoon is used during a pulmonary vein isolation (PVI) procedure, it is important that the cryoballoon completely occludes blood flow from the pulmonary vein to be isolated. If this is the case, then the application of cryo energy could reasonably result in electrically isolating the pulmonary vein.

The efforts that have been made to develop cryoballoon catheters to better adapt to the uneven surface topography present in the left atrium of the heart in order to improve patient outcomes have not been altogether successful. These attempts include using a balloon catheter that claims to better conform to uneven surface anatomy by applying pressure on the catheter shaft to press the balloon assembly into left atrial tissue.

Cryoballoon catheters typically have an inner and outer inflatable balloon. For a given balloon material, balloon burst pressure is related to the wall thickness of the balloon. Balloon wall thickness is directly related to deflated balloon profile. The deflated balloon profile of a balloon catheter often determines the inner diameter (ID) of the delivery sheath through which the balloon catheter must be inserted to be positioned within the heart. It is desirable to reduce the balloon wall thickness of a balloon catheter to further decrease the ID of the delivery sheath used to perform the ablation procedure. Therefore, there remains an unmet need for a dual balloon catheter offering improved operation while offering a reduced profile (providing for a smaller access hole in the living body) compatible with smaller diameter delivery devices.

The inner and outer inflatable balloons of cryoballoon catheters can be bonded in various ways. However, none of these structures describes a conformable, low-profile dual balloon catheter. Low-profile in this instance means a smaller outer diameter (OD) device which enables a smaller access hole into the living body.

To date, commercially available cryogenic dual balloon catheters have utilized adhesively bonded inner and outer inflatable balloons. While it is theoretically possible to use a heat bond to secure an outer inflatable balloon to an inner inflatable balloon, the separate functional requirements of the inner and outer inflatable balloon will be necessarily compromised. This undesirable consequence is due to the inner and outer inflatable balloon having completely different functions. Hence, there remains an unmet need for a low-profile dual balloon assembly offering optimal balloon functional properties.

Obtaining a robust adhesive bond between two balloons requires a long bond joint length, the mating surface area between the inner and outer inflatable balloon, and clean bonding surfaces. Adhesive bond joints are stiff and tend to be larger in diameter than thermal bond joints. These physical attributes make it difficult for catheters to be maneuvered inside the left atrium. As a consequence, adhesively bonded balloon catheters can compromise maneuverability and reduce the likelihood of a successful procedure.

Further, in a typical intravascular catheter system, the cryoballoons are relatively non-compliant and are of a single diameter when in the ablation mode. However, human pulmonary vein diameter and shape can vary significantly within and between patients. Consequently, current cryoballoons offer an all or nothing capability in treating pulmonary veins in pulmonary vein isolation procedures.

Thus, a cryoballoon that is more adaptable to common variations in human pulmonary vein diameter and shape is desired in order to better achieve pulmonary vein occlusion and isolation in a greater percentage of patients treated. Additionally, it is further desired that the change from one balloon outer diameter to another using the same balloon should be achievable multiple times in a predictable fashion. This feature would enable the operator to move the balloon catheter from one pulmonary vein to the next, change the outer diameter of the balloon to occlude the pulmonary vein, apply therapy to achieve a successful outcome, and then move to the next pulmonary vein to repeat the process.

### SUMMARY

The present invention is directed toward a balloon catheter for an intravascular catheter system. In certain embodiments, the balloon catheter includes an outer inflatable balloon and an inner inflatable balloon. The outer inflatable balloon has an outer balloon diameter that is measured at a nominal working balloon pressure. The inner inflatable balloon is positioned substantially within the outer inflatable balloon. The inner inflatable balloon has an inner balloon diameter that is measured at the nominal working balloon pressure. In various embodiments, the inner balloon diameter is greater than the outer balloon diameter at the nominal working balloon pressure.

In some non-exclusive embodiments, the inner balloon diameter is at least approximately 5%, 10%, 15%, 20%, 25% or 30% greater than the outer balloon diameter.

In certain embodiments, the inner balloon diameter can be between approximately 29-35 millimeters, and the outer balloon diameter can be between approximately 23-29 millimeters.

In various embodiments, the inner inflatable balloon can be less compliant than the outer inflatable balloon.

In certain embodiments, the inner inflatable balloon is formed from one or more of polyether block amides and polyurethane. In some such embodiments, the outer inflatable balloon can be formed from one or more of polyether block amides and polyurethane.

In some embodiments, during inflation of the balloon catheter at least a portion of an outer surface of the inner inflatable balloon expands and is positioned substantially directly adjacent to a portion of an inner surface of the outer inflatable balloon.

In various embodiments, the outer inflatable balloon has an outer balloon compliance over a working range, and the inner inflatable has an inner balloon compliance over the working range. In some such embodiments, the inner balloon compliance is less than the outer balloon compliance.

In certain non-exclusive embodiments, the outer balloon compliance is at least approximately 2%, 5%, 8%, 10%, 15% or 20% greater than the inner balloon compliance.

In some embodiments, the inner inflatable balloon can be formed from one of a non-compliant and a semi-compliant material, and the outer inflatable balloon can be formed from one of a semi-compliant and a compliant material.

In another embodiment, the balloon catheter includes an outer inflatable balloon having an outer balloon compliance over a working range, and an inner inflatable balloon that is positioned substantially within the outer inflatable balloon. The inner inflatable balloon has an inner balloon compliance over the working range that is less than the outer balloon compliance.

In yet another embodiment, the present invention is directed toward a balloon catheter that includes a catheter shaft, an inner inflatable balloon that is connected to the catheter shaft, and an outer inflatable balloon that is connected to the catheter shaft. In certain embodiments, the inner inflatable balloon and the outer inflatable balloon are heat-bonded to one another.

In some embodiments, the inner inflatable balloon is heat-bonded to the catheter shaft. In certain embodiments, the outer inflatable balloon is heat-bonded to the catheter shaft.

In various embodiments, the balloon catheter can also include a guidewire lumen that is at least partially positioned within the catheter shaft. In some such embodiments, the inner inflatable balloon can be heat-bonded to the guidewire lumen. In certain embodiments, the outer inflatable balloon can also or alternatively be heat-bonded to the guidewire lumen. In certain embodiments, the outer balloon can also be heat-bonded to the inner balloon.

In certain embodiments, the inner inflatable balloon is formed from one of a non-compliant and a semi-compliant material. In some such embodiments, the outer inflatable balloon is formed from a material having a greater compliance than the material that forms the inner inflatable balloon.

The outer inflatable balloon and the inner inflatable balloon form a dual-balloon assembly. In some such embodiments, the dual-balloon assembly has a distal neck that is tipless.

The present invention is also directed toward a method for manufacturing a balloon catheter for an intravascular catheter system. In certain embodiments, the method includes the step of heat-bonding an inner inflatable balloon and an outer inflatable balloon to one another.

In some embodiments, the step of heat-bonding includes the inner inflatable balloon having less compliance than the outer inflatable balloon.

The method can also include the step of heat-bonding the inner inflatable balloon to a catheter shaft of the balloon catheter.

In some embodiments, the method can include the step of heat-bonding the outer inflatable balloon to a catheter shaft of the balloon catheter.

In various embodiments, the outer inflatable balloon can have a burst pressure that is less than a burst pressure of the inner inflatable balloon.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic view illustration of a patient and one embodiment of an intravascular catheter system having features of the present invention;

FIG. 2 is a side view of a portion of one embodiment of the intravascular catheter system including a balloon catheter;

FIG. 3A is a cross-sectional view of one embodiment of a portion of the balloon catheter including a dual balloon assembly, shown in an extended position;

FIG. 3B is a cross-sectional view of the portion of the balloon catheter including the dual balloon assembly illustrated in FIG. 3A, shown in a retracted position;

FIG. 3C is a cross-sectional view of another embodiment of a portion of the balloon catheter including a dual balloon assembly, shown in an extended position;

FIG. 3D is a cross-sectional view of the portion of the balloon catheter including the dual balloon assembly illustrated in FIG. 3C, shown in a retracted position;

FIG. 4A illustrates a simplified side view of an anatomical region of a body and an embodiment of the dual balloon assembly, shown in the retracted position;

FIG. 4B illustrates a simplified side view of another anatomical region of the body and the dual balloon assembly illustrated in FIG. 4A, shown in the retracted position;

FIG. 4C illustrates a simplified side view of yet another anatomical region of the body and the dual balloon assembly illustrated in FIG. 4A, shown in the retracted position;

FIG. 5A is a graph of one representative embodiment showing balloon catheter compliance including outer diameter as a function of pressure;

FIG. 5B is a table of one representative embodiment showing balloon catheter compliance including outer diameter as a function of pressure;

FIG. 6A is a graph of one representative embodiment showing balloon compliance measurement after five cycles including outer diameter as a function of pressure;

FIG. 6B is a graph of one representative embodiment showing balloon compliance measurement after ten cycles including outer diameter as a function of pressure;

FIG. 6C is a graph of one representative embodiment showing post hysteresis cycling compliance measurement including outer diameter as a function of pressure;

FIG. 6D is a table of one representative embodiment showing balloon compliance measurements for post-hysteresis cycling after five cycles and after ten cycles;

FIG. 7A is a graph of one representative embodiment showing catheter 161 outer diameter hysteresis comparison including outer diameter as a function of pressure;

FIG. 7B is a graph of one representative embodiment showing catheter 162 outer diameter hysteresis comparison including outer diameter as a function of pressure;

FIG. 7C is a graph of one representative embodiment showing catheter 163 outer diameter hysteresis comparison including outer diameter as a function of pressure;

FIG. 7D is a graph of one representative embodiment showing outer diameter hysteresis discrepancy including outer diameter discrepancy as a function of pressure;

FIG. 7E is a table of one representative embodiment showing hysteresis measurements including outer diameter as a function of pressure; and

FIG. 7F is a graph of one representative embodiment showing outer diameter discrepancy including outer diameter discrepancy as a function of pressure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Embodiments of the present invention are described herein in the context of a variable-diameter compliant balloon for use within an intravascular catheter system. More specifically, in various embodiments, the cryoballoons used within the intravascular catheter system are configured to enable the cryoballoons to be selectively adjustable in diameter so as to be more effectively usable within pulmonary veins of different sizes.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of energy can be used to ablate diseased heart tissue. These can include radio frequency (RF), ultrasound, DC pulsed electroporation, and laser energy, as non-exclusive examples. The present invention is intended to be effective with any or all of these and other forms of energy.

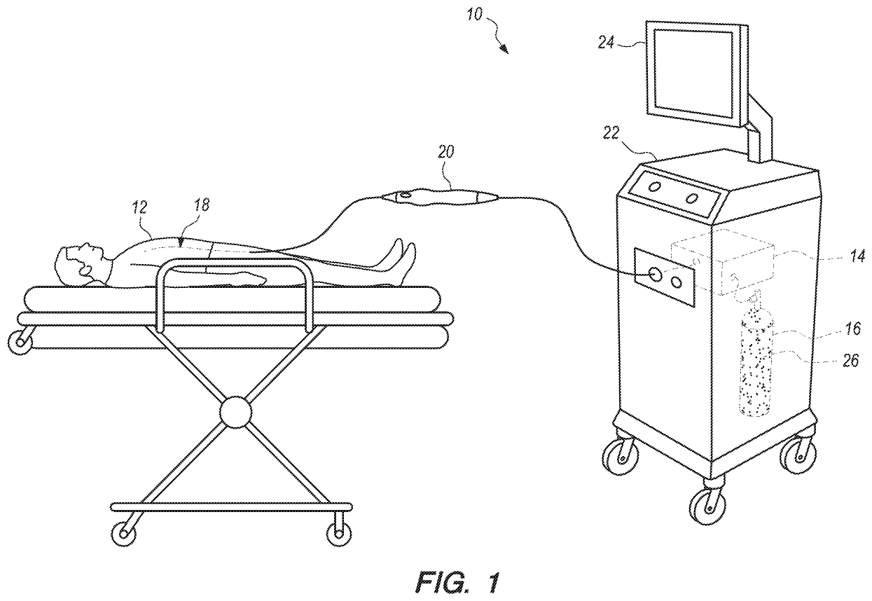

FIG. 1 is a simplified schematic side view illustration of an embodiment of an intravascular catheter system 10 for use with a patient 12, which can be a human being or an animal. The design of the intravascular catheter system 10 can be varied. In certain embodiments, such as the embodiment illustrated in FIG. 1, the intravascular catheter system 10 can include one or more of a control system 14 (illustrated in phantom), a fluid source 16 (illustrated in phantom), a balloon catheter 18, a handle assembly 20, a control console 22, and a graphical display 24.

It is understood that although FIG. 1 illustrates the structures of the intravascular catheter system 10 in a particular position, sequence and/or order, these structures can be located in any suitably different position, sequence and/or order than that illustrated in FIG. 1. It is also understood that the intravascular catheter system 10 can include fewer or additional components than those specifically illustrated and described herein.

In various embodiments, the control system 14 is configured to monitor and control various processes of the ablation procedure. More specifically, the control system 14 can monitor and control release and/or retrieval of a cooling fluid 26 (e.g., a cryogenic fluid) to and/or from the balloon catheter 18. The control system 14 can also control various structures that are responsible for maintaining and/or adjusting a flow rate and/or pressure of the cryogenic fluid 26 that is released to the balloon catheter 18 during the cryoablation procedure. In such embodiments, the intravascular catheter system 10 delivers ablative energy in the form of cryogenic fluid 26 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18. Further, or in the alternative, the control system 14 can receive data and/or other information (hereinafter sometimes referred to as “sensor output”) from various structures within the intravascular catheter system 10. In some embodiments, the control system 14 can receive, monitor, assimilate and/or integrate the sensor output and/or any other data or information received from any structure within the intravascular catheter system 10 in order to control the operation of the balloon catheter 18. As provided herein, in various embodiments, the control system 14 can initiate and/or terminate the flow of cryogenic fluid 26 to the balloon catheter 18 based on the sensor output. Still further, or in the alternative, the control system 14 can control positioning of portions of the balloon catheter 18 within the body of the patient 12, and/or can control any other suitable functions of the balloon catheter 18.

The fluid source 16 contains the cryogenic fluid 26, which is delivered to the balloon catheter 18 with or without input from the control system 14 during a cryoablation procedure. Once the ablation procedure has initiated, the cryogenic fluid 26 can be delivered and the resulting gas, after a phase change, can be retrieved from the balloon catheter 18, and can either be vented or otherwise discarded as exhaust. Additionally, the type of cryogenic fluid 26 that is used during the cryoablation procedure can vary. In one non-exclusive embodiment, the cryogenic fluid 26 can include liquid nitrous oxide. However, any other suitable cryogenic fluid 26 can be used. For example, in one non-exclusive alternative embodiment, the cryogenic fluid 26 can include liquid nitrogen.

The design of the balloon catheter 18 can be varied to suit the specific design requirements of the intravascular catheter system 10. As shown, the balloon catheter 18 is configured to be inserted into the body of the patient 12 during the cryoablation procedure, i.e. during use of the intravascular catheter system 10. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by a healthcare professional (also referred to herein as an “operator”). As used herein, a healthcare professional and/or an operator can include a physician, a physician's assistant, a nurse and/or any other suitable person and/or individual. In certain embodiments, the balloon catheter 18 is positioned within the body of the patient 12 utilizing at least a portion of the sensor output that is received by the control system 14. For example, in various embodiments, the sensor output is received by the control system 14, which can then provide the operator with information regarding the positioning of the balloon catheter 18. Based at least partially on the sensor output feedback received by the control system 14, the operator can adjust the positioning of the balloon catheter 18 within the body of the patient 12 to ensure that the balloon catheter 18 is properly positioned relative to targeted cardiac tissue (not shown).

The handle assembly 20 is handled and used by the operator to operate, position and control the balloon catheter 18. The design and specific features of the handle assembly 20 can vary to suit the design requirements of the intravascular catheter system 10. In the embodiment illustrated in FIG. 1, the handle assembly 20 is separate from, but in electrical and/or fluid communication with the control system 14, the fluid source 16, and the graphical display 24. In some embodiments, the handle assembly 20 can integrate and/or include at least a portion of the control system 14 within an interior of the handle assembly 20. It is understood that the handle assembly 20 can include fewer or additional components than those specifically illustrated and described herein.

In various embodiments, the handle assembly 20 can be used by the operator to initiate and/or terminate the cryoablation process, e.g., start the flow of the cryogenic fluid 26 to the balloon catheter 18 in order to ablate certain targeted heart tissue of the patient 12. In certain embodiments, the control system 14 can override use of the handle assembly 20 by the operator. Stated in another manner, in some embodiments, the control system 14 can terminate the cryoablation process without the operator using the handle assembly 20 to do so.

The control console 22 is coupled to the balloon catheter 18 and the handle assembly 20. Additionally, in the embodiment illustrated in FIG. 1, the control console 22 includes at least a portion of the control system 14, the fluid source 16, and the graphical display 24. However, in alternative embodiments, the control console 22 can contain additional structures not shown or described herein. Still alternatively, the control console 22 may not include various structures that are illustrated within the control console 22 in FIG. 1. For example, in certain nonexclusive alternative embodiments, the control console 22 does not include the graphical display 24.

In various embodiments, the graphical display 24 is electrically connected to the control system 14. Additionally, the graphical display 24 provides the operator of the intravascular catheter system 10 with information that can be used before, during and after the cryoablation procedure. For example, the graphical display 24 can provide the operator with information based on the sensor output and any other relevant information that can be used before, during and after the cryoablation procedure. The specifics of the graphical display 24 can vary depending upon the design requirements of the intravascular catheter system 10, or the specific needs, specifications and/or desires of the operator.

In one embodiment, the graphical display 24 can provide static visual data and/or information to the operator. In addition, or in the alternative, the graphical display 24 can provide dynamic visual data and/or information to the operator, such as video data or any other data that changes over time, e.g., during an ablation procedure. Further, in various embodiments, the graphical display 24 can include one or more colors, different sizes, varying brightness, etc., that may act as alerts to the operator. Additionally, or in the alternative, the graphical display 24 can provide audio data or information to the operator.

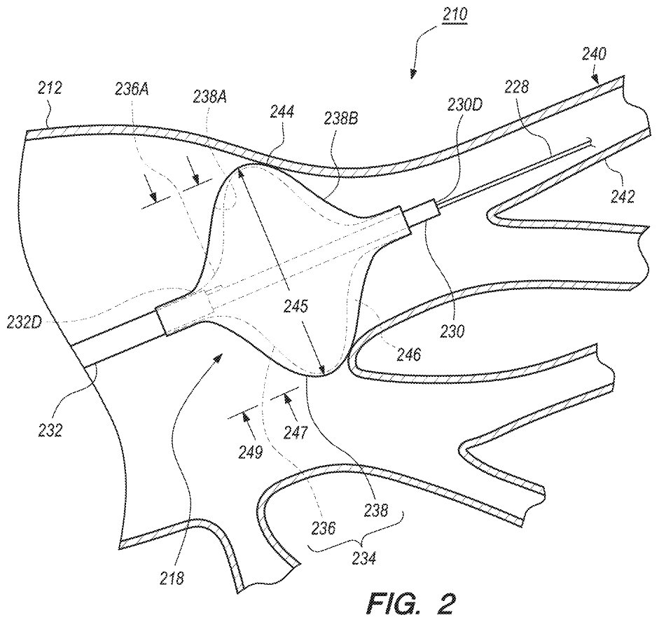

FIG. 2 is a simplified schematic side view of a portion of the patient 212 and a portion of one embodiment of the intravascular catheter system 210. In this embodiment, the intravascular catheter system 210 includes a balloon catheter 218. As an overview, and as provided in greater detail herein, in various embodiments, the intravascular catheter system 210 can include a low-profile, anatomy-conforming balloon catheter 218 (hereinafter referred to as “balloon catheter”) for cryogenically or thermally ablating tissue surrounding one or more pulmonary veins for the treatment of atrial fibrillation in order to improve outcomes and procedural safety. Certain embodiments of the intravascular catheter system 210 can also or alternatively provide a structure able to be delivered through a small profile delivery device.

The design of the balloon catheter 218 can be varied to suit the design requirements of the intravascular catheter system 210. In the embodiment illustrated in FIG. 2, the balloon catheter 218 includes one or more of a guidewire 228, a guidewire lumen 230, a catheter shaft 232, and a dual balloon assembly 234 including an inner inflatable balloon 236 (illustrated in phantom in FIG. 2, and sometimes referred to herein as a “first inflatable balloon”) and an outer inflatable balloon 238 (sometimes referred to herein as a “second inflatable balloon”). As used herein, it is recognized that either inflatable balloon 236, 238 can be described as the first inflatable balloon or the second inflatable balloon. Additionally, it is understood that the balloon catheter 218 can include other structures as well. However, for the sake of clarity, these other structures have been omitted from the Figures. As shown in the embodiment illustrated in FIG. 2, the balloon catheter 218 is configured to be positioned within the circulatory system 240 of the patient 212. The guidewire 228 and guidewire lumen 230 are inserted into a pulmonary vein 242 of the patient 212, and the catheter shaft 232 and the inflatable balloons 236, 238 are moved along the guidewire 228 and/or the guidewire lumen 230 to near an ostium 244 of the pulmonary vein 242. [0069] In various embodiments, the inner inflatable balloon 236 and the outer inflatable balloon 238 can be configured to perform their intended functions and/or to have a somewhat similar physical footprint. In at least some embodiments, this is accomplished using thermal fusing techniques, rather than adhesive bonding techniques. These techniques are possible because of the use of materials in both the inner inflatable balloon 236 and the outer inflatable balloon 238 to enhance compatibility for fusing while preserving the respective functional requirements, of each balloon 236, 238, that are rather different from one another.

In at least some embodiments, the outer inflatable balloon 238 can be heat-bonded to the inner inflatable balloon 236. The outer inflatable balloon 238 can provide for a significantly larger volume, when inflated, than the inner inflatable balloon 236. The intravascular catheter system 210 includes a balloon catheter 218 that provides safer operation by using an outer inflatable balloon 238 having a burst pressure equal to or lower than that of the inner inflatable balloon 236 due to the ability of the outer inflatable balloon 238 to expand to a much greater volume than the inner inflatable balloon 236.

In various embodiments, the inner inflatable balloon 236 can be relatively non-compliant or semi-compliant. A non-compliant or semi-compliant balloon is defined herein as one that inflates to a predetermined shape, and changes to this shape are relatively insensitive to the internal inflation pressure. The inner inflatable balloon 236 can be made from a variety of commonly used materials for use with balloon catheters 218. Materials such as Nylon, Pebax®, Polyurethanes, PET, and Hytrel™ are examples of materials that are suitable for this application. Nylon-12 (Vestamid®, ML-21) and Pebax® in grades 6333, 7033, and 7233 are suited for use in this application.

In various embodiments, the inner inflatable balloon 236 is heat-bonded to the catheter shaft 232 to achieve a small diameter about the catheter shaft 232, using a laser or clam shell heated die set, for example. To facilitate the heat bond, the catheter shaft 232 and the inner inflatable balloon 236 are chosen so they are compatible for heat bonding. Thus, if a shaft distal end 232D of the catheter shaft 232 is made from Pebax® 3533, then the inner inflatable balloon 236 and/or the outer inflatable balloon 238 material choice can include materials that are compatible for heat bonding to this type of catheter shaft 232. In certain embodiments, any Pebax® or Nylon can be a candidate balloon material. Pebax® is a nylon block co-polymer, softer than Nylon but heat bondable to Nylon.

The material choice for the inner inflatable balloon 236 can be selected to provide for a thin walled balloon that resists changing from a designed shape when pressurized. One advantage of a non-compliant or semi-compliant inner inflatable balloon 236 is that it can be formed in very thin walled thicknesses and withstand high internal pressures with only a slight change in shape until it bursts. A non-compliant or semi-compliant inner inflatable balloon 236 can withstand high pressures before bursting, providing for an additional margin of safety against rupture. A nominal balloon burst pressure of approximately 25 psi is suitable for this application, although higher and/or lower designed burst pressures can also be used.

In addition to a high burst pressure and thin walls, there can be other salient features of the inner inflatable balloon 236. These features can include one or more of consistency, robustness, pinhole resistance, low hysteresis, small balloon wrap profile, and small balloon rewrap profile. The inner inflatable balloon 236 provided herein blends these various characteristics so that the final dual balloon catheter 223 has an improved set of performance attributes.

In one embodiment, the inner inflatable balloon 236 can be made as follows. A candidate material such as Nylon-12 is purchased from a supplier, typically in 44 pound bags from single lot number providing traceability of the raw material, an important medical device consideration. The material is provided in small pellets (not shown). The pellets are dried in a sealed chamber (not shown) with a desiccant bed while circulated air, at an elevated temperature, is passed through the pellets to achieve a dew point well below zero degrees Fahrenheit. This increases the likelihood that the raw material used to make the inner inflatable balloon 236 is dry and that moisture is not present during the tubing extrusion process.

The pellets are loaded through a hopper (not shown) into an extrusion system (not shown). Those knowledgeable in the art are aware of such extrusion systems, which can vary and yet still produce balloon tubing capable of making specification conforming inner inflatable balloons 236. The extrusion system utilizes a screw, a metal rod with spiral elements, which turns inside a barrel. A three-quarter inch diameter screw or a one inch diameter screw results in clean extrusion tubing suitable for making an inner inflatable balloon 236 for this application.

Polymer pellets are fed into the extrusion screw, which rotates the pellets into a melt. There are numerous heater bands placed along the path of the pellets. The heated pellets are elevated to temperatures approaching their melt point. The action of the heaters and screw in turn mixes the pellets to homogenize the melt, thus resulting in a clean film melt that is relatively free from imperfections. Because the inner inflatable balloon 236 is designed to have extremely thin walls, excellent homogeneity of the melt is necessary to avoid flaws in the film that lead to premature balloon burst pressures or other undesirable defects.

The melted polymer mix exits the extrusion die set, which is the tooling that shapes the balloon tubing, which is pulled across a small air gap, then is passed into a water filled trough. The water filled trough quickly solidifies the tubing, helping to provide for tubing dimensions and properties that facilitate the balloon forming process. The tubing for the outer inflatable balloon 238 is made in a similar fashion using a material choice ideally suited for the functional needs of the outer inflatable balloon 238.

A variety of extrusion systems and extrusion parameters can be used to arrive at a balloon tubing of ideal properties. The diameter of the extrusion die set is chosen to properly size the inner and outer diameter of the tubing, providing for a draw-down ratio that results in a tubing elongation suitable for balloon forming. The extrusion system may have a cross-head design to provide for uniform back pressure of the melt and extruded tubing. Air pressure provided through the hypotube serves to support the extruded tubing inner diameter. Likewise, screen packs, a stack of open metal screens of multiple micron sized openings, capture contamination and provide added back pressure. Lastly, a pulley system incorporating a laser microscope that works in concert with a puller can achieve and control outer tubing dimensions to ensure the designed balloon wall thickness is met. Variations to this process can still result in tubing suitable for an ideal inner inflatable balloon 236 and/or outer inflatable balloon 238.

In one embodiment, the outer inflatable balloon 238 is made in a somewhat similar fashion to the inner inflatable balloon 236. However, it is made from a mixture of materials that traditionally are not heat bondable. By mixing these disparate materials with widely differing polymeric compositions, an outer inflatable balloon 238 of certain characteristics and that is also heat bondable to an inner inflatable balloon 236 of certain characteristics can be made. Outer inflatable balloons 238 made from balloon tubing in this fashion lend themselves to heat fusing to the inner inflatable balloon 236 or the catheter shaft 232. In constructing a dual balloon assembly 234 from this combination of materials, a relatively small outer diameter, shorter bond joint length and softer bond joint can be fabricated. The benefits of this dual balloon assembly 234 are lower balloon catheter retraction forces into a delivery sheath, improved balloon bond reliability, and lower manufacturing costs.

In one embodiment, the outer inflatable balloon 238 can be made from a mixture of polyurethane, such a Pellethane® 2363-90A TPU (Lubrizol Life Sciences) and Pebax® 6333, a polyamide block copolymer. The ratio of polyurethane to the polyamide block copolymer may be from approximately 10:90 (10% polyurethane to 90% polyamide block copolymer) to approximately 20:80 (20% polyurethane to 80% polyamide block copolymer). The ratio of the mixture can be adjusted to approximately 20-40% polyurethane and approximately 60-80% polyamide block copolymer. The ratio of the mixture can also be adjusted to approximately 40-60% of each material. Alternatively, the ratio of the mixture can also be adjusted to approximately 50% of each material. The different ratios are chosen to improve various balloon performance parameters while still enabling heat fusing of the outer inflatable balloon 238 and inner inflatable balloon 236 together. Inflatable balloons 236, 238, made from these materials enable bonding without the use of adhesives.

The inner inflatable balloon 236 and the outer inflatable balloon 238 can be formed using a balloon forming machine (not shown). Initially the balloon tubing may undergo a stretching process called necking. An eighteen inch segment of balloon tubing is stretched (necked). The two end sections of the balloon tubing are heated to a temperature that softens the tubing and enables stretching of the heated section while not stretching the unheated middle segment. Thus, a small segment in the center of the tubing is left unstretched. This unstretched middle segment, called the parison, will be blow molded into a balloon.

The necked balloon tubing is blow formed into a balloon using a balloon forming machine. The balloon forming machine is comprised of a balloon mold, movable clamps, a pressurized line, and a control system that adjusts and regulates gas pressure inside the balloon tubing and, also, the temperature of the mold. The stretched section of the tubing is reduced in diameter so it can easily be passed through the end of a mold within a balloon forming machine. The forming process will cycle through various temperatures and pressures to heat and soften the balloon tubing, and stretch and pressurize the tubing to expand the parison into one of the inflatable balloons 236, 238. The formed inflatable balloon 236, 238, may subsequently be heat processed in a final step to stabilize the balloon size. Finally, the inflatable balloon 236, 238, is cooled while still pressurized to under 100 degrees Fahrenheit to prevent unwanted shrinking. When the inflatable balloon 236, 238, is cooled to under 100 degrees Fahrenheit, the mold is opened and the pressure inside the inflatable balloon 236, 238, is released. The inflatable balloon 236, 238, is then extracted from the mold.

In certain embodiments, the outer inflatable balloon 238 fits snugly over the inner inflatable balloon 236 so there are no gaps or spaces between the inner inflatable balloon 236 and the outer inflatable balloon 238. This helps ensure maximal thermal transfer between the outer inflatable balloon 238 and tissue by reducing undesirable air pockets or wrinkles between the two inflatable balloons 236, 238. A technique to reduce the air gaps and wrinkles between inner inflatable balloon 236 and the outer inflatable balloon 238 is to shrink the outer inflatable balloon 238, using a heat process, onto a fully formed inner inflatable balloon 236. The process to manufacture this assembly is as follows: The outer inflatable balloon 238 is first formed without a final heat process, known as balloon annealing, which under typical balloon forming processes is used to stabilize the balloon from shrinkage. Without the final annealing process, the outer inflatable balloon 238 can be shrunk a fully formed and completed inner inflatable balloon 236 component using heat. After shrinking the outer inflatable balloon 238 onto the inner inflatable balloon 236, both the inner inflatable balloon 236 and the outer inflatable balloon 238 are annealed together in a mold to stabilize the balloon diameter using the annealing process, thus preferentially shrinking only the outer inflatable balloon 238 onto a stable and shrink resistant inner inflatable balloon 236. This annealing process stabilizes the balloon assembly size enabling it to withstand temperature excursions during subsequent manufacturing processes, such as ethylene oxide sterilization. During some of these processes, the inner inflatable balloon 236 can be internally pressurized to prevent it from shrinking.

To enhance utility of the balloon catheter 218, the shape and size of the inner inflatable balloon 236 and/or the outer inflatable balloon 238 can vary. Anticipated shapes include a disc like shape with a diameter of approximately 26-32 mm. Larger and smaller diameters can also be used. In addition, tubular, pear-like and other balloon shapes can be included in the present invention.

The balloon catheter 218 described herein may also include electrodes (not shown) affixed to the inner inflatable balloon 236 and/or the outer inflatable balloon 238. In one embodiment, the electrodes may be configured as part of a flex circuit array. The flex circuit array can be configured so that the electrodes are spaced evenly apart or spaced in bipolar pairs or quadripolar arrays, where two or four electrodes are mounted relatively close to each other and then separated by a relatively larger distance. In one embodiment, a spacing arrangement can be a center-to-center interelectrode distance of 2 mm, center-to-center, or 1 mm edge-to-edge. The spacing between bipolar pairs of electrodes may be 3, 4, or 5 mm. The same spacing pattern can be applied to the quadripolar array. In the example of mounting the electrodes on the inner inflatable balloon 236, the outer inflatable balloon 238 will be made to be conductive. Conductive balloons can be made using conductive additives, such as carbon, to typically non-conductive polymers commonly used for balloon materials. Additionally, in one embodiment, the outer balloon can be conductive in only a z-axis direction. This can be accomplished using anisotropically conductive material applied within the outer balloon film or locally directly over the electrodes.

As provided herein, one way to treat a wider range of human anatomy is to better size the inflatable balloons 236, 238 of the balloon catheter 218 to encompass and/or match a diameter of the pulmonary vein 242. In general, it is the object of the balloon catheter 218 to seal the pulmonary vein 242 so that blood flow is occluded. Only when occlusion is achieved does the cryogenic energy, e.g., the cryogenic fluid 26 (illustrated in FIG. 1), cause tissue necrosis which, in turn, provides for electrically blocking aberrant electrical signals that trigger atrial fibrillation. Unfortunately, as noted above, human anatomy varies, and the diameter of pulmonary veins varies within a given patient as well as between patients.

As an overview, in various embodiments as described in detail herein, one way to treat the variety of pulmonary vein diameters is to provide a balloon catheter 218 that includes inflatable balloons 236, 238 that are selectively adjustable to provide a range of overall diameters 245. Based on the varying diameters of the pulmonary veins in the human body, the ideal range of an overall diameter 245 of an inflated dual balloon assembly 234 may range from approximately 26 to 32 mm, although it is understood that the true value for the diameter of any given pulmonary vein can vary outside the normal parameters thus potentially requiring an overall diameter 245 that may be greater than 32 mm or less than 26 mm. Accomplishing the desired range of overall diameters 245, however, is not trivial. In conventional inflatable balloons in current use, there is a lack of inflatable balloon materials that lend themselves to meet all the performance and safety requirements for a cryoballoon and enable a useful range of overall diameters 245. For example, non-compliant inflatable balloons (described herein as inflatable balloons with less than approximately 6% compliance over working range) or semi-compliant inflatable balloons (described herein as inflatable balloons with approximately 6-12% compliance over working range) in general use typically do not offer a wide enough range of overall diameters 245 to meet the clinical need. Conversely, while compliant inflatable balloons (described herein as inflatable balloons with greater than approximately 12% compliance over working range) made from very soft polymers expand readily to fit the anatomy, they are plagued by hysteresis and have low burst pressures that fail to offer appropriate levels of safety.

Thus, in various embodiments, the present invention is directed toward a balloon catheter 218 that includes the inner inflatable balloon 236 that is less compliant than the outer inflatable balloon 238. In various embodiments described herein, the inner inflatable balloon 236 has an inner balloon diameter 247 and the outer inflatable balloon 238 has an outer balloon diameter 249. In various embodiments, taken separately (not as a dual balloon assembly 234) the inner balloon diameter 247 is greater than the outer balloon diameter 249 at a nominal working balloon pressure. In other words, when the inflatable balloons 236, 238, are separated from one another and are not assembled together in the dual balloon assembly 234, at a given nominal pressure the inner balloon diameter 247 is greater than the outer balloon diameter 249. When the inflatable balloons are together in the dual balloon assembly 234, the inner balloon diameter 247 is substantially the same as the outer balloon diameter 249 since the outer inflatable balloon 238 substantially conforms the shape and/or size of the inner inflatable balloon 236 when the balloons 236, 238, are inflated. As used herein, in certain applications, the nominal working balloon pressure can be between approximately 1.5 psi and 3.5 psi. More specifically, in one application, the nominal working balloon pressure can be approximately 2.5 psi. Alternatively, in other applications, the nominal working balloon pressure can be greater than 3.5 psi or less than 1.5 psi.

For example, in some such embodiments, the inner inflatable balloon 236 can be non-compliant or semi-compliant and have an inner balloon diameter 247 that is between approximately 29 mm and 35 mm at a nominal working balloon pressure, and the outer inflatable balloon 238 can be more compliant and have an outer balloon diameter 249 that is between approximately 23 mm and 29 mm at a nominal working balloon pressure. As noted, in various embodiments, the inner inflatable balloon 236 is less compliant than the outer inflatable balloon 238, and/or the inner balloon diameter 247 is larger than the outer balloon diameter 249 at a nominal working balloon pressure.

In certain non-exclusive embodiments, the inner balloon diameter 247 can at a given nominal working balloon pressure can be between approximately 0% and 30% greater than the outer balloon diameter 249 at the same nominal working balloon pressure. For example, the inner balloon diameter 247 at a nominal working balloon pressure can be at least approximately 1%, 2%, 3%, 5%, 7%, 10%, 12%, 15%, 17%, 20%, 22%, 25%, 27% or 30% greater than the outer balloon diameter 249 at the same nominal working balloon pressure. Alternatively, the inner balloon diameter 247 at a given nominal working balloon pressure can be greater than 30% greater than the outer balloon diameter 249 at the same nominal working balloon pressure.

Additionally, in some non-exclusive embodiments, the inner inflatable balloon 236 can be relatively non-compliant or semi-compliant and can have a compliance over a nominal working range of less than approximately 1%, 2%, 3%, 4%, 5%, 6%, 7%, 8%, 9%, 10%, 11%, 12%, 13%, 14%, 15%, 16%, 17%, 18%, 19% or 20%. Further, in certain non-exclusive embodiments, the outer inflatable balloon 238 can be relatively semi-compliant or compliant and can have a compliance over a nominal working range of at least approximately 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%.

Moreover, in some non-exclusive embodiments, the compliance of the outer inflatable balloon 238 can be between approximately 1% and 20% greater than the compliance of the inner inflatable balloon 236. For example, in such embodiments, the compliance of the outer inflatable balloon 238 can be at least approximately 1%, 2%, 3%, 4%, 5%, 6%, 7%, 8%, 9%, 10%, 11%, 12%, 13%, 14%, 15%, 16%, 17%, 18%, 19% or 20% greater than the compliance of the inner inflatable balloon 236. Alternatively, the compliance of the outer inflatable balloon 238 can be more than 20% greater than the compliance of the inner inflatable balloon 236.

In such embodiments, by combining a less compliant inner inflatable balloon 236 that has a relatively large inner balloon diameter 247, e.g., 32 mm at a nominal working balloon pressure of approximately 2.5 psi for example, and a more compliant outer inflatable balloon 238 having an outer balloon diameter 249 that is relatively small in comparison to the inner balloon diameter 247, e.g., 26 mm at a nominal working balloon pressure of approximately 2.5 psi, the outer inflatable balloon 238 can somewhat constrain expansion of the inner inflatable balloon 238 during and after inflation. This combination further enables a balloon catheter 218 with a wide range of overall diameters 245 within a small range of working pressures to increase the likelihood of safe, low-pressure operation that decreases the chances of a balloon rupture, which can lead to patient injury and death.

With this design, the range of available overall diameters 245 that can be achieved is increased. In addition, constraining an upper limit to the overall diameter 245 of the dual balloon assembly 234 with the less compliant inner inflatable balloon 236 effectively puts a ceiling on overall diameter 245 increase. Further, the inner inflatable balloon 236 has a higher burst pressure and contributes more in protecting against an inadvertent rupture.

As illustrated, the inner inflatable balloon 236 is positioned substantially, if not completely, within the outer inflatable balloon 238. It is appreciated that the relatively large inner balloon diameter 247 of the inner inflatable balloon 236 may have to be folded or otherwise manipulated to fit within the relatively small outer inflatable balloon 238. However, as the inflatable balloons 236, 238 are inflated, e.g., as the inner inflatable balloon 236 is directly inflated, which then indirectly increases the size of the outer inflatable balloon 238, such folds or manipulations of the inner inflatable balloon 236 do not adversely impact the desired operation of the inner inflatable balloon 236, and may be only temporary.

As noted, the smaller outer inflatable balloon 238 constrains the inner inflatable balloon 236 from expanding to its nominal size at low pressures by having a relatively small outer balloon diameter 249. Only at higher pressures does the hoop stress inside the inner inflatable balloon 236 overcome the constraining forces of the outer inflatable balloon 238 to increase the overall diameter 245 of the dual-balloon assembly 234. Optimally, the characteristics of each of the inner inflatable balloon 236 and the outer inflatable balloon 238, including the inner balloon diameter 247, the outer balloon diameter 249, a balloon wall thickness for each of the inner inflatable balloon 236 and the outer inflatable balloon 238, and balloon material selection for each of the inner inflatable balloon 236 and the outer inflatable balloon 238 are chosen to reduce balloon hysteresis, provide for an adequate range of expansion, and offer a high burst pressure relative to the working pressure range of the intravascular catheter system 210.

The specific design of and materials used for each of the inner inflatable balloon 236 and the outer inflatable balloon 238 can be varied. In certain embodiments, the inner inflatable balloon 236 can be made from relatively non-compliant or semi-compliant materials. Additionally, the inner inflatable balloon 236 will typically be formed to the high end of a working diameter range. For example, in one non-exclusive embodiment, for an intravascular catheter system 210 capable of spanning from 26 mm to 32 mm, the diameter of the inner inflatable balloon 236 at a nominal working balloon pressure can be approximately 32 mm, though it may be more or less to achieve desirable diameter ranges and accommodate post balloon forming processes such as sterilization which may shrink the balloon. Further, in some embodiments, the inner inflatable balloon 236 is bonded to a shaft distal end 232D of the catheter shaft 232 and near a lumen distal end 230D of the guidewire lumen 230. A variety of bonding techniques can be used and can include heat bonding and/or adhesive bonding.

A lubricious biocompatible material such as a grease may be inserted between the inflatable balloons 236, 238, to enable free expansion against the constraining outer inflatable balloon 238. Other suitable lubricants can alternatively be used. Still alternatively, a lubricious additive may be compounded into either the inner inflatable balloon tubing or outer inflatable balloon tubing to reduce friction between the two inflatable balloons 236, 238, during inflation to better enable predictable and repeatable balloon diameters for a given pressure. The lubricant increases the likelihood that the intended balloon diameter is achieved at the various pressures defining the working range, such as 26 to 32 mm inflated balloon diameter. The lubricant can also reduce the working pressures, as far as is possible, so that the full working range of balloon diameter may be several multiples below the burst pressure of the dual balloon assembly 234. For example, a dual balloon assembly 234 may have an average burst pressure of 30 psi. A working range of pressures such as 2.5 psi to 11 psi ensures that there is a significant margin of safety between the balloon burst pressure and the pressure range needed to provide the full span of balloon diameters that the operator may desire.

After the two inflatable balloons 236, 238 are bonded to the catheter shaft 232, the intravascular catheter system 210 can be completed. After assembly, the inflatable balloons 236, 238 may be subjected to at least one inflation cycle to reduce hysteresis in the system. The completed device is then sterilized using ETO gas, for example. Additionally, in one embodiment, either of the inflatable balloons 236, 238, may be rendered electrically conductive by doping the material from which it is made with a conductive metal or other conductive substance. In such embodiment, the electrically conductive inflatable balloons can be particularly suitable for the outer inflatable balloon 238.

During use, the inner inflatable balloon 236 can be partially or fully inflated so that at least a portion of the inner inflatable balloon 236 expands against at least a portion of the outer inflatable balloon 238. Stated in another manner, during use of the balloon catheter 218, at least a portion of an outer surface 236A of the inner inflatable balloon 236 expands and is positioned substantially directly against a portion of an inner surface 238A of the outer inflatable balloon 238. At certain times during usage of the intravascular catheter system 210, the inner inflatable balloon 236 and the outer inflatable balloon 238 define an inter-balloon space 246, or gap, between the inflatable balloons 236, 238. The inter-balloon space 246 is illustrated between the inner inflatable balloon 236 and the outer inflatable balloon 238 in FIG. 2 for clarity, although it is understood that at certain times during usage of the intravascular catheter system 210, the inter-balloon space 246 has very little volume. As provided herein, once the inner inflatable balloon 236 is sufficiently inflated, an outer surface 238B of the outer inflatable balloon 238 can then be positioned within the circulatory system 240 of the patient 212 to abut and/or substantially form a seal with the ostium 244 of the pulmonary vein 242 to be treated.

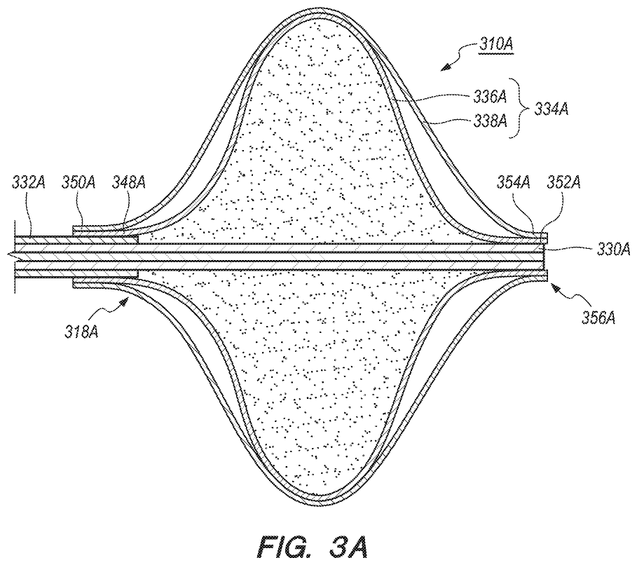

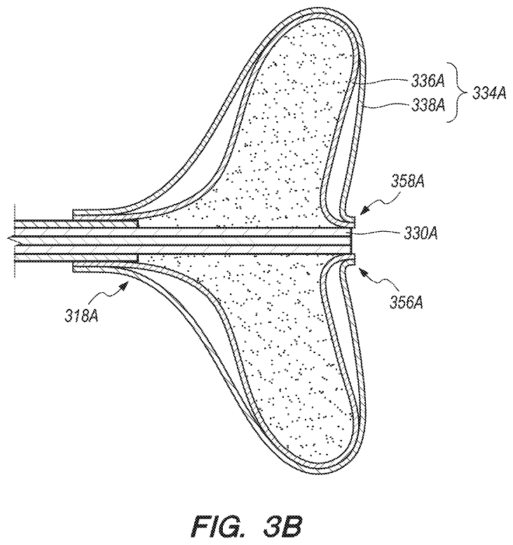

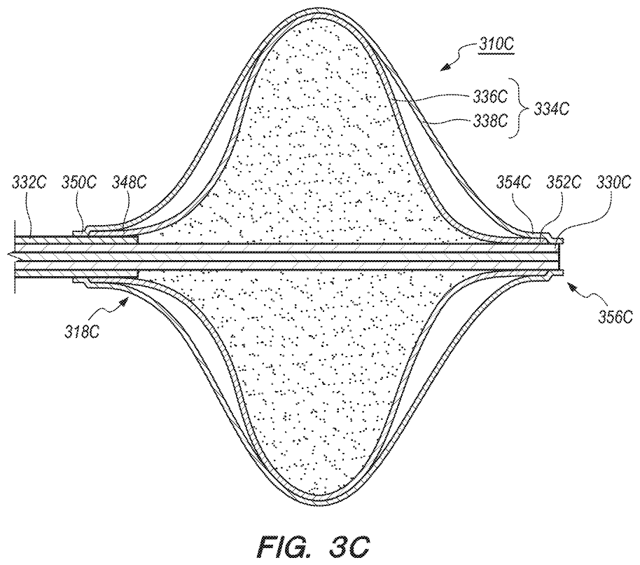

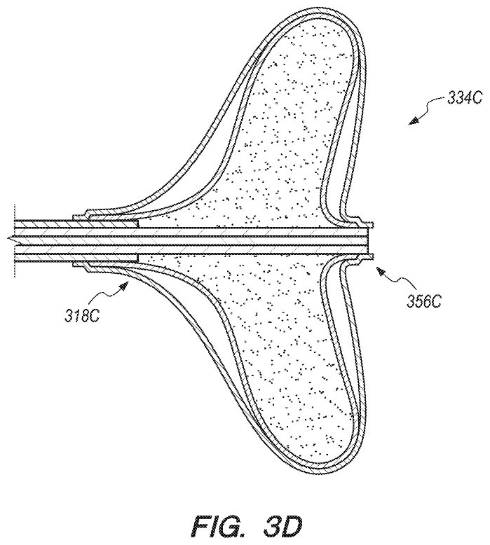

FIG. 3A is a cross-sectional view of one embodiment of a portion of an intravascular catheter system 310A. The intravascular catheter system 310A includes a portion of the balloon catheter 318A that is somewhat similar to the balloon catheter 218 illustrated in FIG. 2. In this embodiment, the balloon catheter 318 includes a dual balloon assembly 334A, shown in an extended position. For the sake of clarity, it is understood that the balloon catheters illustrated herein may include additional structures not shown and/or described, or may omit various structures that are shown and/or described.

The dual balloon assembly 334A includes an inner inflatable balloon 336A and an outer inflatable balloon 338A which can be substantially similar to those previously described herein. In the embodiment illustrated in FIG. 3A, once the inner inflatable balloon 336A and the outer inflatable balloon 338A have been formed, the balloons 336A, 338A, can be heat-bonded to the catheter shaft 332A in a variety of ways. A proximal neck 348A of the inner inflatable balloon 336A can be heat-bonded directly onto the catheter shaft 332A. In the embodiment illustrated in FIG. 3A, a proximal neck 350A of the outer inflatable balloon 338A can be heat-bonded to the proximal neck 348A of the inner inflatable balloon 336A. Further, in this embodiment, a distal neck 352A of the inner inflatable balloon 336A can be heat-bonded to the guidewire lumen 330A. In the embodiment illustrated in FIG. 3A, a distal neck 354A of the outer inflatable balloon 338A can be heat-bonded to the distal neck 352A of the inner inflatable balloon 336A. In the extended position, the distal neck 354A of the outer inflatable balloon 338A is extended outwardly to a form a distal tip 356A of the balloon catheter 318A. The guidewire lumen 330A is moved distally (further into the patient 12), which pulls the dual balloon assembly 334A along with the guidewire lumen 330A to form the distal tip 356A.

The preferred method of these bonding procedures for either or both of the inner inflatable balloon 336A and outer inflatable balloon 338A is heat-bonding using a clamshell style heated die set or a laser bonder. Other suitable heat-bonding techniques can also be used. Though less desirable due to increased overall thickness, the inner inflatable balloon 336A and/or the outer inflatable balloon 338A can also be bonded using more traditional methods such as adhesive bonding. A variety of adhesive formulations are suitable for this application including cyanoacrylate, UV or LED curable polyoligomer adhesives, as well as two part epoxy adhesive formulations. One non-exclusive example of an adhesive is Dymax 204 CTH.

FIG. 3B is a cross-sectional view of the portion of the balloon catheter 318A including the dual balloon assembly 334A illustrated in FIG. 3A, shown in a retracted position. The dual balloon assembly 334A includes an inner inflatable balloon 336A and an outer inflatable balloon 338A. One benefit of a heat-bonded dual balloon assembly 334A for use in a balloon catheter 318A is that the heat-bonding, due to its inherent flexibility, is amenable to retraction to form a distal catheter end 358A without a very small or even non-existent distal tip 356A, thereby allowing the dual balloon assembly 334A to move to the retracted position. A retracted dual balloon assembly 334A can more easily maneuver around left atrial anatomy, and can better comply with and/or match the left atrial anatomy (or other locations in and around the heart) compared with the dual balloon assembly 334A in the extended position, or with another type of balloon catheter that cannot move to the retracted position, as the added length of the distal tip 356A would otherwise make the balloon catheter 318A difficult to position and move within the left atrium of the heart.

In an alternative embodiment, the dual balloon assembly 334A can always be in the retracted position, and cannot be moved to the extended position. This type of dual balloon assembly 334A is sometimes referred to herein as a “tipless dual balloon assembly”. In one embodiment, the tipless dual balloon assembly 334A can be made using the above described inner inflatable balloon 336A and outer inflatable balloon 338A, assembled in tandem or individually to the catheter shaft 332A and/or guidewire lumen 330A. The guidewire lumen 330A can be retracted into the catheter shaft 332A and secured into the handle of the cryoballoon such that when the balloon is inflated the distal catheter end 358A is aligned with the distal neck 354A of the outer inflatable balloon 338A. The alignment of the distal catheter end 358A may be fully retracted into the outer inflatable balloon 338A and/or the inner inflatable balloon 336A. This structure provides a relatively compact shape, eliminating the approximately 8 to 13 mm tip from the total length of the dual-balloon assembly 334A. Moreover, the reduction and/or elimination of the distal tip 356A and/or the distal catheter end 358A enables treatment at sites other than the pulmonary veins where a distal tip 356A would inhibit contact between the outer inflatable balloon 338A and cardiac tissue of the patient 12 (illustrated in FIG. 1).

FIG. 3C is a cross-sectional view of one embodiment of an intravascular catheter system 310C. The intravascular catheter system 310C includes a portion of the balloon catheter 318C including a dual balloon assembly 334C, shown in an extended position. The dual balloon assembly 334C includes an inner inflatable balloon 336C and an outer inflatable balloon 338C. In the embodiment illustrated in FIG. 3C, once the inner inflatable balloon 336C and the outer inflatable balloon 338C have been formed, the balloons 336C, 338C, can be heat-bonded to the catheter shaft 332C in a variety of ways. A proximal neck 348C of the inner inflatable balloon 336C can be heat-bonded directly onto the catheter shaft 332C. In the embodiment illustrated in FIG. 3C, a proximal neck 350C of the outer inflatable balloon 338C can be heat-bonded to the proximal neck 348C of the inner inflatable balloon 336C and/or directly to the catheter shaft 332C. Further, in this embodiment, a distal neck 352C of the inner inflatable balloon 336C can be heat-bonded to the guidewire lumen 330C. A distal neck 354C of the outer inflatable balloon 338C can be heat-bonded to the distal neck 352C of the inner inflatable balloon 336C and/or directly to the guidewire lumen 330C. In the extended position, the distal neck 354C of the outer inflatable balloon 338C is extended outwardly to a form a distal tip 356C of the balloon catheter 318C. The guidewire lumen 330C is moved distally (further into the patient 12), which pulls the dual balloon assembly 334C along with the guidewire lumen 330C to form the distal tip 356C.

FIG. 3D is a cross-sectional view of the portion of the balloon catheter 318C including the dual balloon assembly 334C illustrated in FIG. 3C, shown in the retracted position. Somewhat similar to that previously described herein, the retracted dual balloon assembly 334C can more easily maneuver around left atrial anatomy compared with the dual balloon assembly 334C in the extended position, or with another type of balloon catheter that cannot move to the retracted position, as the added length of the distal tip 356C would otherwise make the balloon catheter 318C difficult to position and move within the left atrium of the heart. In an alternative embodiment, the dual balloon assembly 334C can always be in the retracted position, and cannot be moved to the extended position.

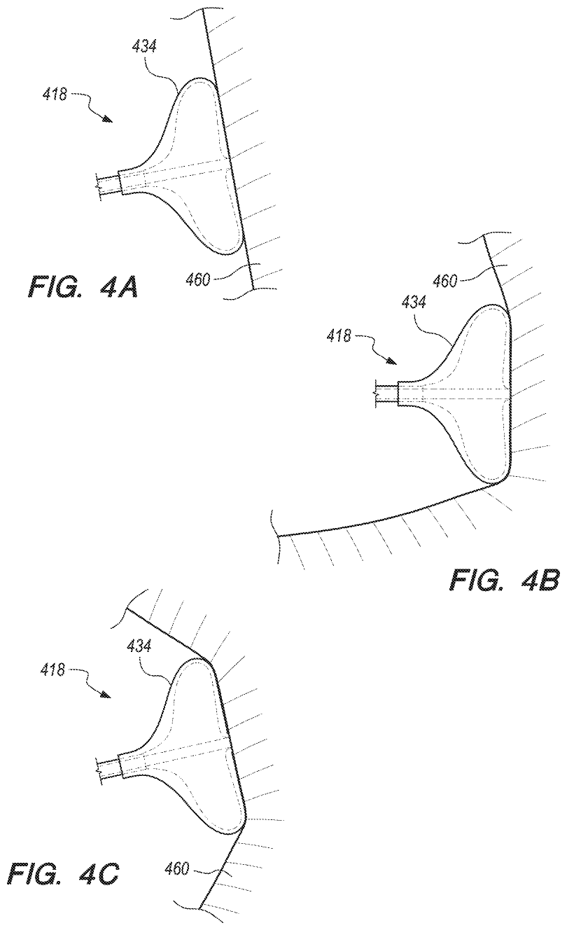

FIGS. 4A-4C illustrate how the balloon catheter 418 having a dual balloon assembly 434 in the retracted position (as illustrated in FIGS. 3B and 3D, for example) can conform to portions of cardiac and/or vascular anatomy 460 of a patient 12 (illustrated in FIG. 1). For example, portions of the posterior wall, the left atrial roofline, and portions of the anterior wall of the left atrium, as non-exclusive examples, can be better ablated. The dual balloon assemblies 434 illustrated and described herein enable spot ablations at non-pulmonary vein sites as described above. Alternatively, linear ablations, a series of connected ablations extending from a section within the left atrium to another site in the left atrium, can be better achieved. Other heart chambers including the right atrium and left and right ventricles can also be treated using the dual balloon assemblies 434 illustrated and described herein.

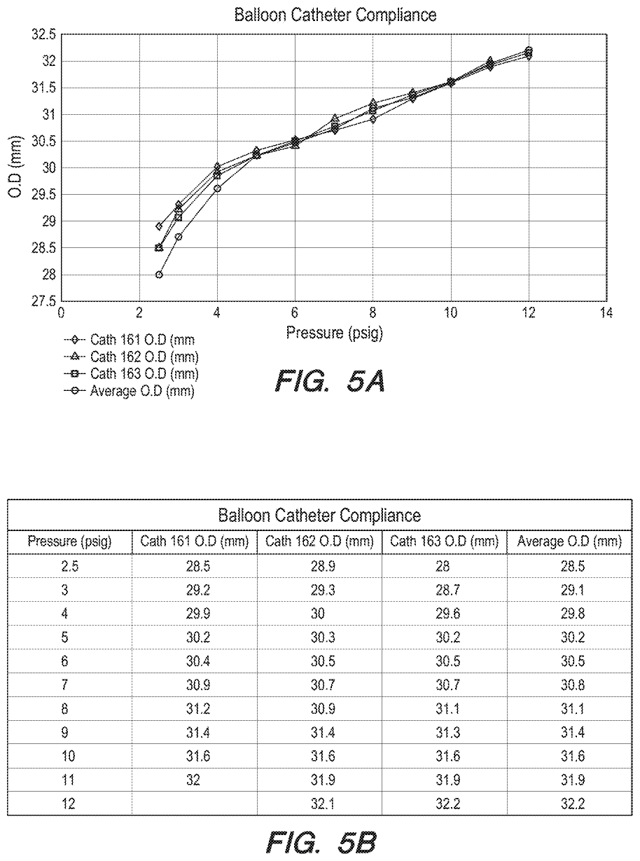

FIG. 5A is a graph of one representative embodiment of the dual-balloon assembly showing balloon catheter compliance including an outer diameter (also referred to herein as “overall diameter”) in millimeters as a function of balloon pressure (in psig) in three separate dual-balloon assemblies (identified as 161, 162 and 163). The graph in FIG. 5A illustrates that the overall diameter of the dual-balloon assemblies, on average, is approximately 28 mm at 2.5 psig and increases to approximately 32 mm at 12 psig.

FIG. 5B is a table of one representative embodiment of the dual-balloon assembly showing balloon catheter compliance including outer diameter (also referred to herein as “overall diameter”) in millimeters as a function of balloon pressure (in psig) in three separate dual-balloon assemblies (identified as 161, 162 and 163), and the average of the three separate dual-balloon assemblies. The table in FIG. 5B illustrates that the overall diameter of the dual-balloon assemblies, on average, is approximately 28 mm at approximately 2.5 psig and increases to approximately 32 mm at approximately 12 psig.

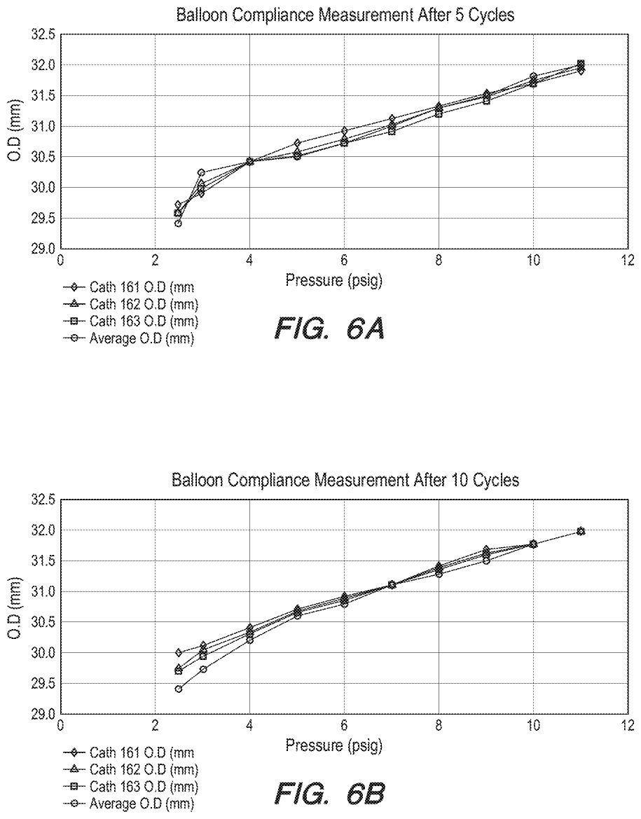

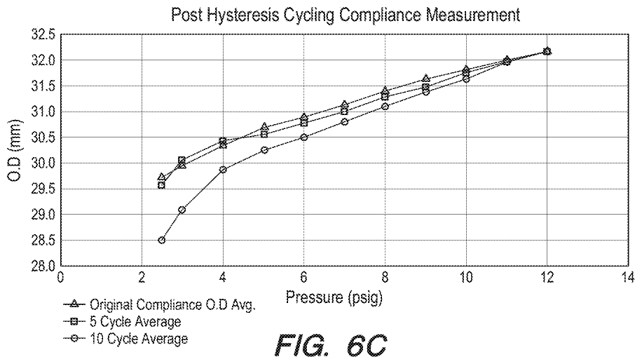

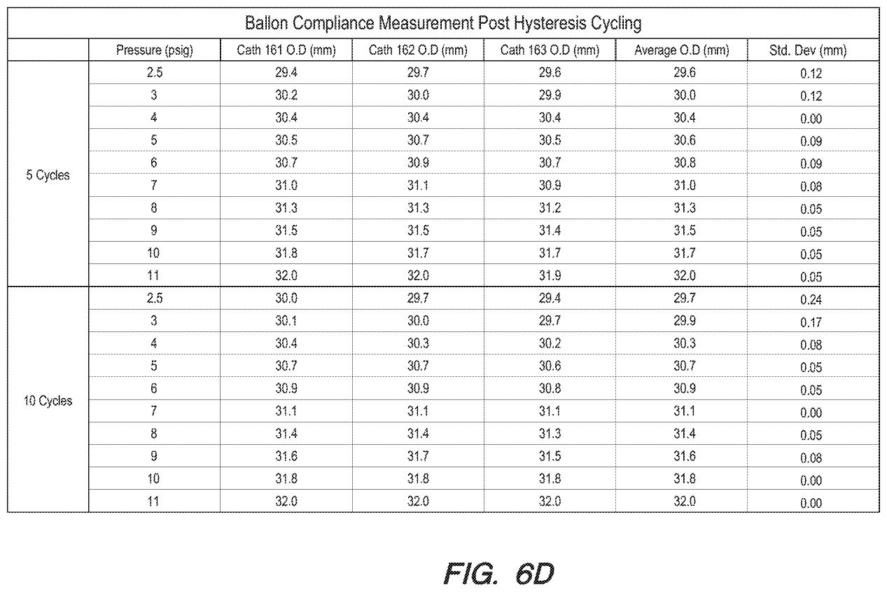

FIG. 6A is a graph of one representative embodiment of the dual-balloon assembly showing balloon catheter compliance after five inflation-deflation cycles including overall diameter (in millimeters) as a function of balloon pressure (in psig). The graph in FIG. 6A illustrates that the overall diameter of the dual-balloon assemblies, on average, is approximately 29.5 mm at approximately 2.5 psig and increases to approximately 32 mm at approximately 11 psig.

FIG. 6B is a graph of one representative embodiment of the dual-balloon assembly showing balloon catheter compliance after ten inflation-deflation cycles including overall diameter (in millimeters) as a function of balloon pressure (in psig). The graph in FIG. 6B illustrates that the overall diameter of the dual-balloon assemblies, on average, is approximately 29.5-30.0 mm at approximately 2.5 psig and increases to approximately 32 mm at approximately 11 psig.

FIG. 6C is a graph of one representative embodiment of the dual-balloon assembly showing post hysteresis cycling compliance measurement including outer diameter (in millimeters) as a function of balloon pressure (in psig), including original compliance, a 5 cycle average and a 10 cycle average.

FIG. 6D is a table of one representative embodiment showing balloon compliance measurements for post-hysteresis cycling after five cycles and after ten cycles for three separate dual-balloon assemblies (identified as 161, 162 and 163), including average and standard deviation.

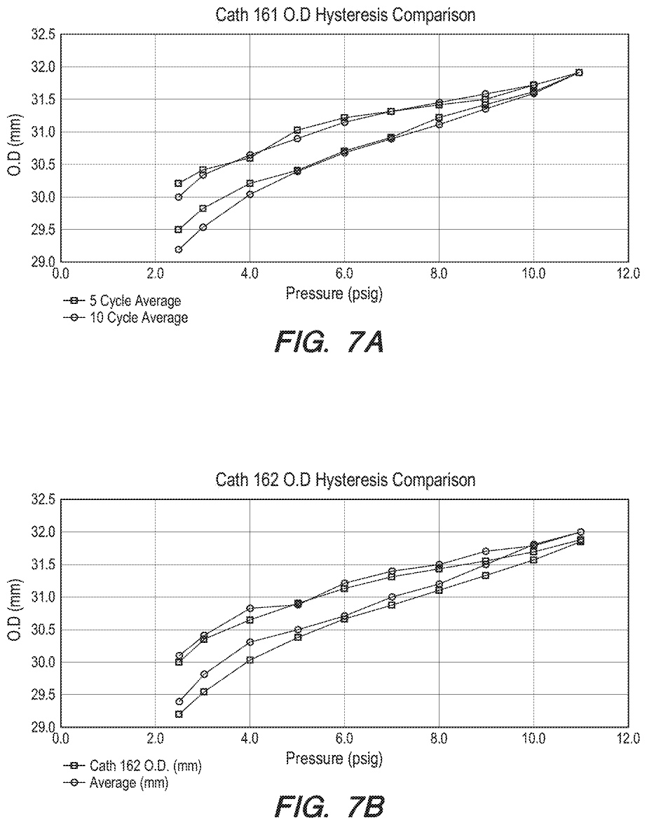

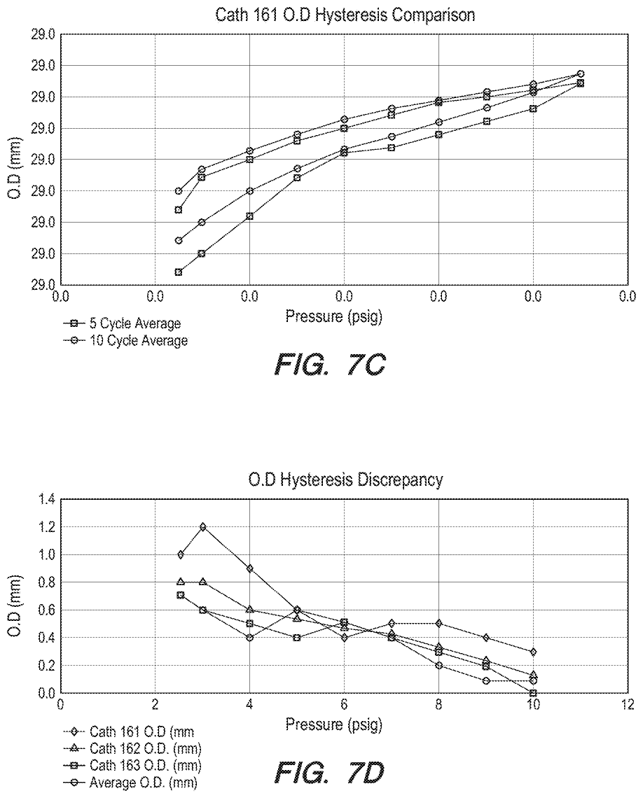

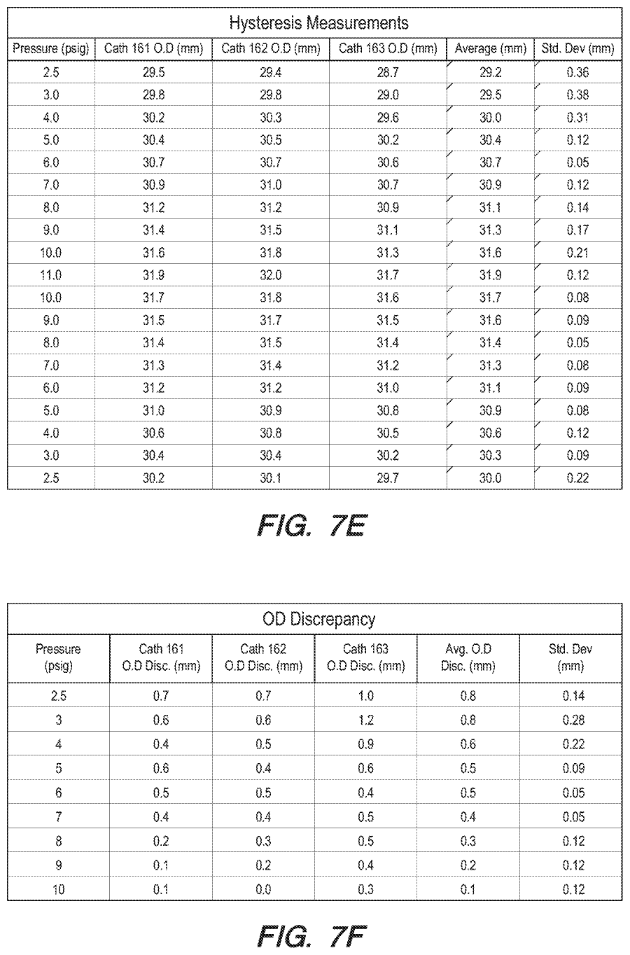

FIG. 7A is a graph of one representative embodiment showing dual-balloon assembly 161 outer diameter hysteresis comparison including outer diameter (in millimeters) as a function of balloon pressure (in psig).

FIG. 7B is a graph of one representative embodiment showing dual-balloon assembly 162 outer diameter hysteresis comparison including outer diameter (in millimeters) as a function of balloon pressure (in psig).

FIG. 7C is a graph of one representative embodiment showing dual-balloon assembly 163 outer diameter hysteresis comparison including outer diameter (in millimeters) as a function of balloon pressure (in psig).

FIG. 7D is a graph of one representative embodiment showing outer diameter hysteresis discrepancy including outer diameter discrepancy (in millimeters) as a function of balloon pressure (in psig) for three separate dual-balloon assemblies (identified as 161, 162 and 163).

FIG. 7E is a table of one representative embodiment showing hysteresis measurements including outer diameter (in millimeters) as a function of balloon pressure (in psig) for three separate dual-balloon assemblies (identified as 161, 162 and 163), including average and standard deviation.

FIG. 7F is a graph of one representative embodiment showing outer diameter discrepancy including outer diameter discrepancy (in millimeters) as a function of balloon pressure (in psig) for three separate dual-balloon assemblies (identified as 161, 162 and 163), including average and standard deviation.

It is understood that although a number of different embodiments of the intravascular catheter system 10 have been illustrated and described herein, one or more features of any one embodiment can be combined with one or more features of one or more of the other embodiments, provided that such combination satisfies the intent of the present invention.

While a number of exemplary aspects and embodiments of the intravascular catheter system 10 have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

I claim:

1. A balloon catheter for an intravascular catheter system, the balloon catheter comprising:
a catheter shaft;
an inner inflatable balloon that is connected to the catheter shaft; and an outer inflatable balloon that is connected to the catheter shaft,
wherein the inner balloon is formed from polyurethane or a polyether block amide, and the outer balloon is formed from a blend of polyurethane and polyether block amide, and
wherein the inner inflatable balloon and the outer inflatable balloon are heat-bonded to one another.

2. The balloon catheter of claim 1, wherein one or both of the inner inflatable balloon and the outer inflatable balloon are heat-bonded to the catheter shaft.

3. The balloon catheter of claim 1, wherein the inner balloon is formed from a polyamide block copolymer.

4. The balloon catheter of claim 3, wherein a ratio of percent polyurethane to percent polyether block amide in the outer balloon is between 10:90 and 20:80.

5. The balloon catheter of claim 3, further comprising a guidewire lumen that is at least partially positioned within the catheter shaft, wherein the inner inflatable balloon is heat-bonded to the guidewire lumen.

6. The balloon catheter of claim 5, wherein the inner inflatable balloon has a proximal neck and a distal neck, and wherein the outer inflatable balloon has a proximal neck and a distal neck, and wherein the proximal neck of the inner inflatable balloon and the proximal neck of the outer inflatable balloon are heat-bonded to the catheter shaft, and wherein the distal neck of the outer inflatable balloon and the distal neck of the inner inflatable balloon are heat-bonded to the guidewire lumen.

7. The balloon catheter of claim 5, wherein the inner inflatable balloon has a proximal neck and a distal neck, and wherein the outer inflatable balloon has a proximal neck and a distal neck, and wherein the proximal neck of the inner inflatable balloon is heat-bonded to the catheter shaft and the proximal neck of the outer inflatable balloon is heat-bonded to the proximal neck of the inner inflatable balloon.

8. The balloon catheter of claim 7, wherein the distal neck of the outer inflatable balloon is heat-bonded to the distal neck of the inner inflatable balloon, and the distal neck of the inner inflatable balloon is heat-bonded to the guidewire lumen.

9. The balloon catheter of claim 8, wherein the outer inflatable balloon has an outer balloon compliance over a working balloon pressure range, and wherein the inner inflatable balloon has an inner balloon compliance over the working balloon pressure range, and wherein the outer balloon compliance is greater than the inner balloon compliance.

10. The balloon catheter of claim 9, wherein the outer balloon compliance is between 2% and 20% greater than the inner balloon compliance over the working pressure range.

11. The balloon catheter of claim 10, wherein in an inflated state at least a portion of an outer surface of the inner inflatable balloon is positioned substantially directly adjacent to a portion of an inner surface of the outer inflatable balloon.

12. The balloon catheter of claim 11, further comprising a biocompatible lubricious material between the outer surface of the inner inflatable balloon and the inner surface of the outer inflatable balloon.

13. The balloon catheter of claim 11, wherein a lubricious additive is compounded into the material forming one or both of the inner inflatable balloon and the outer inflatable balloon.

14. The balloon catheter of claim 1, wherein the outer inflatable balloon and the inner inflatable balloon form a dual-balloon assembly, the dual-balloon assembly having a distal neck that is tipless when the balloon is in a fully inflated state.

15. A balloon catheter for an intravascular catheter system, the balloon catheter comprising:
a catheter shaft having a shaft distal end;
a guidewire lumen at least partially disposed within the catheter shaft and having a lumen distal end disposed distally of the shaft distal end; and
a dual-balloon assembly comprising:
an inner inflatable balloon having a proximal neck heat-bonded to the shaft distal end and a distal neck heat-bonded to the lumen distal end;
an outer inflatable balloon having a proximal neck heat-bonded to the proximal neck of the inner inflatable balloon, and a distal neck heat-bonded to the distal neck of the inner inflatable balloon; and
a biocompatible lubricious material between an outer surface of the inner inflatable balloon and an inner surface of the outer inflatable balloon.

16. The balloon catheter of claim 15, wherein the outer inflatable balloon has an outer balloon compliance over a working balloon pressure range, and wherein the inner inflatable balloon has an inner balloon compliance over the working balloon pressure range, and wherein the outer balloon compliance is between 2% and 20% greater than the inner balloon compliance.

17. The balloon catheter of claim 16, wherein the inner balloon is formed from a polyether block amide, and the outer balloon is formed from a blend of polyurethane and polyether block amide.

18. A method for manufacturing a balloon catheter for an intravascular catheter system, the method comprising heat-bonding an inner inflatable balloon and an outer inflatable balloon to one another.

19. The method of claim 18, wherein the inner inflatable balloon has a proximal neck and a distal neck, and the outer inflatable balloon has a proximal neck and a distal neck, and wherein heat-bonding the inner inflatable balloon and the outer inflatable balloon includes heat-bonding the proximal neck of the inner inflatable balloon and the proximal neck of the outer inflatable balloon, and heat-bonding the distal neck of the inner inflatable balloon and the distal neck of the outer inflatable balloon.

20. The method of claim 18, further comprising heat-bonding the proximal neck of the inner inflatable balloon to a catheter shaft of the balloon catheter.
